(19) United States
(12) Patent Application Publication
(10) Pub. No.: US 2009/0299431 A1
(43) Pub. Date: Dec. 3, 2009

(54) High Voltage Confirmation System Utilizing Impedance Data

(75) Inventor: Stuart O. Schecter, Great Neck, NY (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/130,785

(22) Filed: May 30, 2008

(51) Int. Cl. A61M 1/08

(52) U.S. Cl. 607/28

(57) ABSTRACT

Systems and methods for providing high voltage confirmation are disclosed. In various embodiments, impedance data can be used as a basis for determining the operation of a high voltage confirmation system. In some embodiments, measurements of impedance associated with the high voltage lead(s) can provide indication as to the condition of the lead(s). In some embodiments, faulty leads can yield impedance values that exceed a known threshold value. In some embodiments, such threshold value can be determined from a laboratory study of the leads under conditions that are similar to the operating conditions of implantable cardiac devices.

### FIELD OF THE INVENTION

The present disclosure generally relates to implantable cardiac stimulation devices, and more particularly, to systems and methods for utilizing impedance data for operation of an implantable cardiac device configured to provide high voltage stimulation therapy.

### BACKGROUND OF THE INVENTION

Over the past several decades, large numbers of people have received implanted cardiac stimulation devices such as pacemakers and intra-cardiac defibrillators (ICDs). These devices include leads that are implanted so as to be positioned proximate the walls of the heart, e.g., implanted into the chambers of the heart. These leads typically serve two functions, to deliver therapeutic stimulation to the heart of the patient, and to sense cardiac activity and provide signals indicative thereof to a control unit so that the control unit can determine whether to deliver stimulation to the patient's heart. One problem that can occur over time is that the leads can become partially or fully fractured. In general, the leads are implanted into a very harsh environment where they are subject to repetitive mechanical stress and strain. Over a long time period, the lead can become fractured.

Fully fractured leads are generally incapable of delivering therapeutic stimulation to the heart of the patient. Partially fractured leads also provide problems in that they also may not be efficient at delivering therapeutic stimulation. Further, fractured leads can create noise on the lead. The noise signals may be interpreted by the control unit as indicative of heart activity. In worse case scenarios, the noise signals may be interpreted as a cardiac event that would by indicative of the need for stimulation to be applied to the heart. Consequently, lead fractures can result in the patient receiving heart stimulation when stimulation is not needed.

Unnecessary stimulation can potentially be very harmful to the patient. At a minimum, unnecessary stimulation can result in significant discomfort to the patient. Cardioversion or defibrillation waveforms, when delivered to a conscious patient, can be extremely painful. If the patient is periodically receiving unnecessary stimulations of this sort, the patient's quality of life can be significantly affected. There have been instances where patients have suffered psychological harm as a result of receiving such stimulations.

Certain parameters can be evaluated to assess the performance of a lead and to determine whether the lead has a partial or full fracture. One parameter is to measure the impedance of the lead. One difficulty with measuring impedance is that if the measurement is made at a time when high voltage stimulation is not being provided, e.g., the impedance measurement is made using a low voltage signal, the fracture may not be adequately detected. Often a partial fracture is difficult to detect at very low voltages so the source of noise which may result in inadvertent stimulation of the heart may go undetected.

Based upon the foregoing, there is a need for an improved way of sensing abnormalities with the leads of an implanted device that may result in spurious signals being received by the control unit thereby inducing the delivery of undesired therapeutic stimulations. To this end, there is a need for an analytic framework whereby impedance sensing on the lead may be performed in a manner that will more accurately determine whether there is a fracture or other physical problem with the lead that could be inducing noise.

### SUMMARY

A wide variety of systems, devices, methods, and processes comprising embodiments of the invention are described herein. In various embodiments, impedance data can be used as a basis for determining the operation of a high voltage confirmation system. In some embodiments, measurements of impedance associated with the high voltage lead can provide indication as to the condition of the lead. In some embodiments, faulty lead can yield impedance values that exceed a known threshold value. In some embodiments, such threshold value can be determined from a laboratory study of the lead under conditions that are similar to the operating conditions of implantable cardiac devices.

One embodiment of the invention is a system for differentiating noise from an arrhythmia of a heart, comprising a noise discriminator configured to receive an electrocardiogram (EGM) signal and to discriminate between an organized EGM signal and a chaotic EGM signal based at least in part on an impedance parameter associated with a lead that provides an electrical connection to the heart and a signal analyzer configured to determine whether a chaotic signal is caused by a disturbance in the lead.

Another embodiment is an implantable cardiac device, comprising a high voltage device configured to deliver a therapy signal to a heart when triggered, an electrical lead for connecting the high voltage device to the heart, an impedance measurement component configured to measure an electrical impedance associated with the electrical lead and a processor configured to provide a command for operation of the high voltage device based at least in part on a parameter associated with the measured electrical impedance.

Another embodiment is a method for operating an implantable cardiac device, comprising measuring an impedance value associated with at least one of a plurality of electrical leads for a high voltage device configured to provide a therapy signal, wherein the plurality of electrical leads are configured to be connected to a heart and deliver the therapy signal to the heart and generating a command for operation of the high voltage device based at least in part on the measured impedance value.

Another embodiment is a method for differentiating noise from an arrhythmia of a heart, comprising receiving an electrocardiogram (EGM) signal, measuring an impedance parameter associated with a lead that provides an electrical connection to the heart and determining whether the EGM signal is an organized signal or a chaotic signal based at least in part on the measured impedance parameter.

### BRIEF DESCRIPTION OF THE DRAWINGS

FIG. 1 is a simplified diagram illustrating an implantable stimulation device in electrical communication with at least three leads implanted into a patient's heart for delivering multi-chamber stimulation and shock therapy;

FIG. 2 is a functional block diagram of a multi-chamber implantable stimulation device illustrating the basic elements of a stimulation device which can provide cardioversion, defibrillation and pacing stimulation in four chambers of the heart;

FIG. 3 shows that in some embodiments, a high voltage confirmation system (HVCS) can include an impedance data component;

FIG. 4A shows that in some embodiments, impedance associated with high voltage lead can be analyzed by the HVCS to provide one or more functionalities associated with the operation of the HVCS;

FIG. 4B shows an example of how impedance or impedance related values can be measured with respect to the high voltage lead of the implantable cardiac device;

FIG. 5 shows by way of example that in some embodiments, an impedance value can be monitored and analyzed periodically;

FIG. 6 shows by way of example that in some embodiments, various sampling techniques can be utilized in sampling of impedance related values;

FIG. 7 shows by way of example that in some embodiments, integrated values of impedance can be obtained and analyzed;

FIG. 8 shows an example trend of integrated impedance values that can be monitored and analyzed;

FIG. 9 shows another example trend of integrated impedance values that can be monitored and analyzed;

FIG. 10 shows that in some embodiments, a process can obtain one or more impedance parameters and determine whether to take action based on such parameter(s);

FIG. 11 shows an example process that can perform the process of FIG. 10;

FIGS. 12A-12F show various example actions that can be implemented by the process of FIG. 11;

FIG. 13 shows by way of example how an impedance parameter reference can be formed; and

FIG. 14 shows by way of example how the operation of an implantable cardiac device can be based at least in part on a comparison of a measured impedance-related value with the impedance parameter reference.

These and other aspects, advantages, and novel features of the present teachings will become apparent upon reading the following detailed description and upon reference to the accompanying drawings. In the drawings, similar elements have similar reference numerals.

### DETAILED DESCRIPTION OF SOME EMBODIMENTS

The present disclosure generally relates to a high voltage confirmation system (HVCS) for implantable cardiac stimulation devices. More particularly, various embodiments of the HVCS can include a component configured to utilize one or more impedance and/or impedance-related parameters associated with the operation of the HVCS. Additional details about HVCS are available in a co-pending U.S. application Ser. No. 11/249,684 filed Oct. 12, 2005, titled “Method and Apparatus for Differentiating Lead Noise from Ventricular Arrhythmia” (Attorney Docket No. A05P4001) which is incorporated herein by reference in its entirety. Additional information on how cardiac therapy devices can be programmed to process impedance signals can be found in U.S. Pat. No. 7,010,347 titled “Optimization of Impedance Signals for Closed Loop Programming of Cardiac Resynchronization Therapy Devices” which is incorporated herein by reference in its entirety.

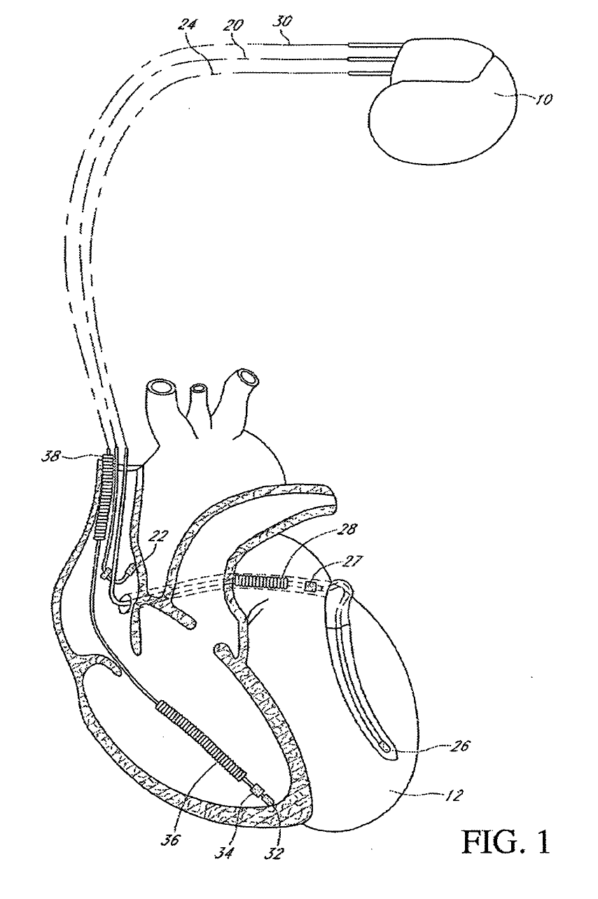

In one embodiment, as shown in FIG. 1, a device 10 comprising an implantable cardiac stimulation device 10 is in electrical communication with a patient's heart 12 by way of three leads, 20, 24 and 30, suitable for delivering multi-chamber stimulation and shock therapy. To sense atrial cardiac signals and to provide right atrial chamber stimulation therapy, the stimulation device 10 is coupled to an implantable right atrial lead 20 having at least an atrial tip electrode 22, which typically is implanted in the patient's right atrial appendage.

To sense left atrial and ventricular cardiac signals and to provide left chamber pacing therapy, the stimulation device 10 is coupled to a “coronary sinus” lead 24 designed for placement in the “coronary sinus region” via the coronary sinus ostium (OS) for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28.

The stimulation device 10 is also shown in electrical communication with the patient's heart 12 by way of an implantable right ventricular lead 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart 12 so as to place the right ventricular tip electrode 32 in the right ventricular apex so that the RV coil electrode will be positioned in the right ventricle and the SVC coil electrode 38 will be positioned in the superior vena cava. Accordingly, the right ventricular lead 30 is capable of receiving cardiac signals, and delivering stimulation in the form of pacing and shock therapy to the right ventricle.

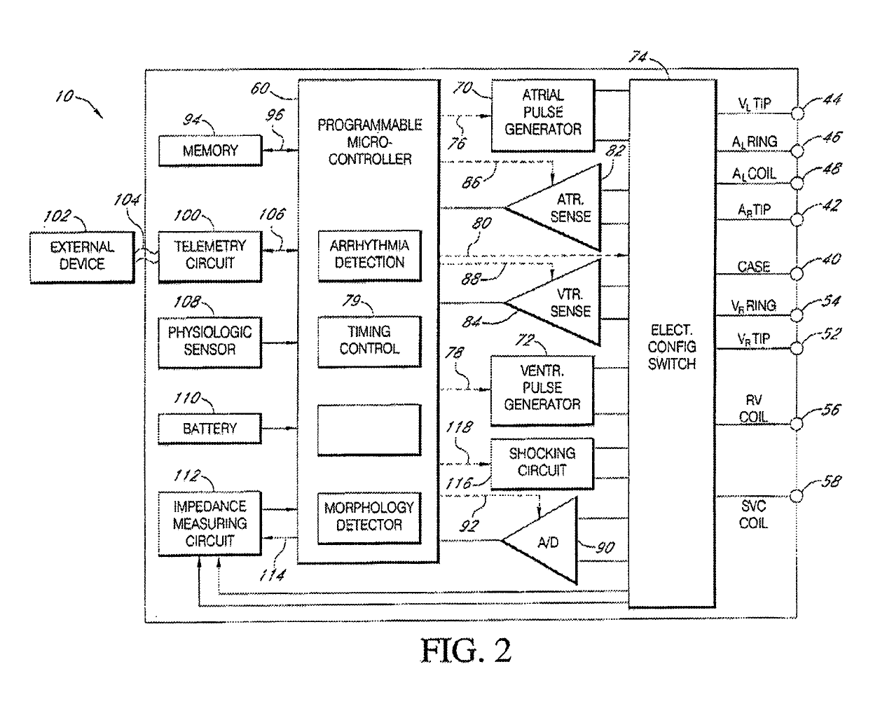

As illustrated in FIG. 2, a simplified block diagram is shown of the multi-chamber implantable stimulation device 10, which is capable of treating both fast and slow arrhythmias with stimulation therapy, including cardioversion, defibrillation, and pacing stimulation. While a particular multi-chamber device is shown, this is for illustration purposes only and one of skill in the art could readily duplicate, eliminate or disable the appropriate circuitry in any desired combination to provide a device capable of treating the appropriate chamber(s) with cardioversion, defibrillation and pacing stimulation.

The housing 40 for the stimulation device 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes, 28, 36 and 38, for shocking purposes. The housing 40 further includes a connector (not shown) having a plurality of terminals, 42, 44, 46, 48, 52, 54, 56, and 58 (shown schematically and, for convenience, the names of the electrodes to which they are connected are shown next to the terminals). As such, to achieve right atrial sensing and pacing, the connector includes at least a right atrial tip terminal (AR TIP) 42 adapted for connection to the atrial tip electrode 22.

To achieve left chamber sensing, pacing and shocking, the connector includes at least a left ventricular tip terminal (VL TIP) 44, a left atrial ring terminal (AL RING) 46, and a left atrial shocking terminal (AL COIL) 48, which are adapted for connection to the left ventricular tip electrode 26, the left atrial ring electrode 27, and the left atrial coil electrode 28, respectively.

To support right chamber sensing, pacing and shocking, the connector further includes a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32, right ventricular ring electrode 34, the RV coil electrode 36, and the SVC coil electrode 38, respectively.

At the core of the stimulation device 10 is a programmable microcontroller 60 which controls the various modes of stimulation therapy. As is well known in the art, the microcontroller 60 typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by a program code stored in a designated block of memory. The details of the design and operation of the microcontroller 60 are not critical to the invention. Rather, any suitable microcontroller 60 may be used that carries out the functions described herein. The use of microprocessor-based control circuits for performing timing and data analysis functions are well known in the art.

As shown in FIG. 2, an atrial pulse generator 70 and a ventricular pulse generator 72 generate pacing stimulation pulses for delivery by the right atrial lead 20, the right ventricular lead 30, and/or the coronary sinus lead 24 via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators, or shared pulse generators. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 which is used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, PVARP intervals, noise detection windows, evoked response windows, alert intervals, marker channel timing, etc., which is well known in the art.

The switch 74 includes a plurality of switches for connecting the desired electrodes to the appropriate I/O circuits, thereby providing complete electrode programmability. Accordingly, the switch 74, in response to a control signal 80 from the microcontroller 60, determines the polarity of the stimulation pulses (e.g., unipolar, bipolar, combipolar, etc.) by selectively closing the appropriate combination of switches (not shown) as is known in the art. In this embodiment, the switch 74 also supports simultaneous high resolution impedance measurements, such as between the case or housing 40, the right atrial electrode 22, and right ventricular electrodes 32, 34 as described in greater detail below.

Atrial sensing circuits 82 and ventricular sensing circuits 84 may also be selectively coupled to the right atrial lead 20, coronary sinus lead 24, and the right ventricular lead 30, through the switch 74 for detecting the presence of cardiac activity in each of the four chambers of the heart. Accordingly, the atrial (ATR. SENSE) and ventricular (VTR. SENSE) sensing circuits, 82 and 84, may include dedicated sense amplifiers, multiplexed amplifiers, or shared amplifiers. The switch 74 determines the “sensing polarity” of the cardiac signal by selectively closing the appropriate switches, as is also known in the art. In this way, the clinician may program the sensing polarity independently of the stimulation polarity.

Each sensing circuit, 82 and 84, preferably employs one or more low power, precision amplifiers with programmable gain and/or automatic gain control, bandpass filtering, and a threshold detection circuit, as known in the art, to selectively sense the cardiac signal of interest. The automatic gain control enables the device 10 to deal effectively with the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation) are then classified by the microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to the inputs of an analog-to-digital (A/D) data acquisition system 90. The data acquisition system 90 is configured to acquire intracardiac electrogram (IEGM) signals, convert the raw analog data into a digital signal, and store the digital signals for later processing and/or telemetric transmission to an external device 102. The data acquisition system 90 is coupled to the right atrial lead 20, the coronary sinus lead 24, and the right ventricular lead 30 through the switch 74 to sample cardiac signals across any pair of desired electrodes.

The microcontroller 60 is further coupled to a memory 94 by a suitable data/address bus 96, wherein the programmable operating parameters used by the microcontroller 60 are stored and modified, as required, in order to customize the operation of the stimulation device 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 12 within each respective tier of therapy.

Advantageously, the operating parameters of the implantable device 10 may be non-invasively programmed into the memory 94 through a telemetry circuit 100 in telemetric communication with the external device 102, such as a programmer, transtelephonic transceiver, or a diagnostic system analyzer. The telemetry circuit 100 is activated by the microcontroller by a control signal 106. The telemetry circuit 100 advantageously allows IEGMs and status information relating to the operation of the device 10 (as contained in the microcontroller 60 or memory 94) to be sent to the external device 102 through an established communication link 104.

In the preferred embodiment, the stimulation device 10 further includes a physiologic sensor 108, commonly referred to as a “rate-responsive” sensor because it is typically used to adjust pacing stimulation rate according to the exercise state of the patient. However, the physiological sensor 108 may further be used to detect changes in cardiac output, changes in the physiological condition of the heart, or diurnal changes in activity (e.g., detecting sleep and wake states). Accordingly, the microcontroller 60 responds by adjusting the various pacing parameters (such as rate, AV Delay, V-V Delay, etc.) at which the atrial and ventricular pulse generators, 70 and 72, generate stimulation pulses.

The stimulation device additionally includes a battery 110 which provides operating power to all of the circuits shown in FIG. 2. For the stimulation device 10, which employs shocking therapy, the battery 110 must be capable of operating at low current drains for long periods of time and then be capable of providing high-current pulses (for capacitor charging) when the patient requires a shock pulse. The battery 110 must also have a predictable discharge characteristic so that elective replacement time can be detected. Accordingly, embodiments of the device 10 including shocking capability preferably employ lithium/silver vanadium oxide batteries. For embodiments of the device 10 not including shocking capability, the battery 110 will preferably be lithium iodide or carbon monoflouride or a hybrid of the two.

As further shown in FIG. 2, the device 10 is shown as having an impedance measuring circuit 112 which is enabled by the microcontroller 60 via a control signal 114.

In the case where the stimulation device 10 is intended to operate as an implantable cardioverter/defibrillator (ICD) device, it must detect the occurrence of an arrhythmia, and automatically apply an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 joules), as controlled by the microcontroller 60. Such shocking pulses are applied to the patient's heart 12 through at least two shocking electrodes, and as shown in this embodiment, selected from the left atrial coil electrode 28, the RV coil electrode 36, and/or the SVC coil electrode 38. As noted above, the housing 40 may act as an active electrode in combination with the RV electrode 36, or as part of a split electrical vector using the SVC coil electrode 38 or the left atrial coil electrode 28 (i.e., using the RV electrode as a common electrode.

Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

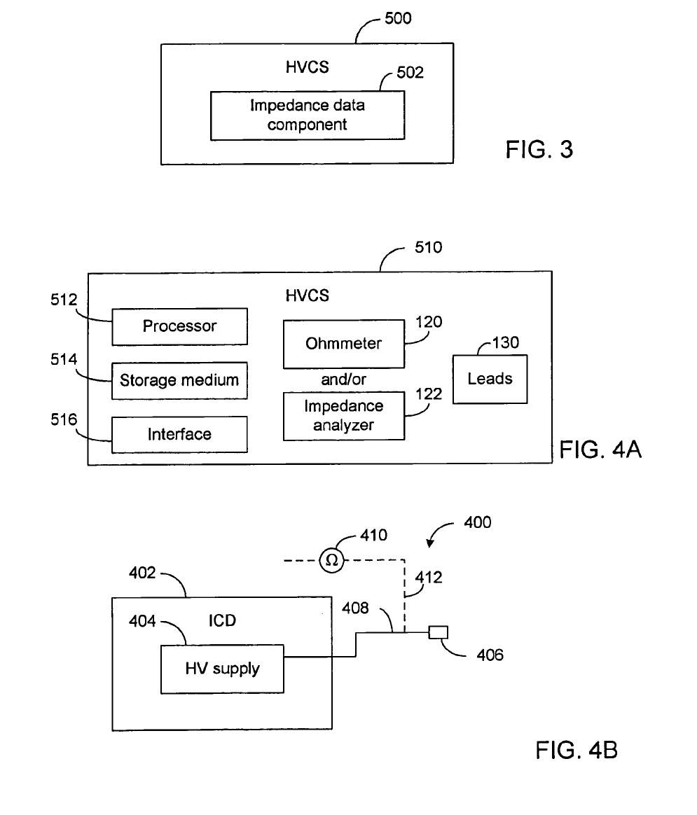

FIG. 3 shows that in some embodiments, a high voltage confirmation system (HVCS) 500 that can be functionally implemented by the micro-controller 60 of an implanted cardiac stimulation device 10, can include an impedance data component 502 configured to facilitate one or more functionalities of the HVCS 500 based on impedance data. In one implementation, the impedance component 502 comprises the impedance measuring circuit 112. For the purpose of description herein, it will be understood that “impedance data” or “impedance” can include impedance value itself and/or a derived value that is based on the impedance value. For example, a sampled impedance value a given time can be impedance data. In another example, an integrated value of impedance over some time interval can also be impedance data. Other derived values are also possible.

In some embodiments, as shown in FIG. 4A, an HVCS 510 can include one or more leads 130 such as the leads 20, 24, 30 of FIG. 1 positioned to deliver high voltage treatment signal(s) to a heart. Such a lead 130 can be electrically connected to electrical components configured to measure resistance (depicted as an ohmmeter 120) or the impedance measuring circuit 112 and/or analyze impedance (depicted as an impedance analyzer 122) associated with the lead 130.

As further shown in FIG. 4A, the HVCS 510 can also include a processor 512 which can be either the microprocessor 60 or a stand-alone processor for processing of impedance data thus obtained (via the example ohmmeter 120 and/or impedance analyzer 122). The HVCS 510 can also include a storage medium 514, such as the memory 94 of the device 10 or a stand-alone memory having one or more processes, reference data, and other data, to facilitate processing of impedance data by the processor 512. In some embodiments, the HVCS 510 can also include an interface 516, such as the telemetry circuit 100 or a stand-alone device, configured to allow interfacing of the HVCS 510 with an external device 102. Such interfacing can include, but not limited to, transfer of impedance data, transfer of reference data, and updating of one or more processes implemented in the implantable cardiac device.

In general, it will be appreciated that processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

FIG. 4B shows examples of how impedance or impedance related values associated with the high voltage lead can be measured. As shown in an example configuration 400, an implantable cardiac device 402 can include a high voltage device 404 configured to deliver high voltage waveform to the heart 12 in the manner discussed above. Such delivery can be effectuated by a high voltage electrode 406, such as the electrodes 28, 36 shown in FIG. 1, via a lead.

In some embodiments, as shown in FIG. 4B, impedance measurement can be made between the electrode 406 and a location (for example, the casing 10) so that the high voltage lead contributes to the resistance between the two measurement points. Thus, an ohmmeter 410 or other impedance measuring circuit 112 can measure an impedance that includes the contribution by the high voltage lead 406 and electrode 408.

In certain situations, degradation of the lead can result in increase in impedance. In certain situations, however, a decrease in impedance can indicate some fault such as a possible short involving the lead or its connections. Thus, in examples described herein, detections of faulty lead can be based on some high and/or low impedance values or other values derived therefrom.

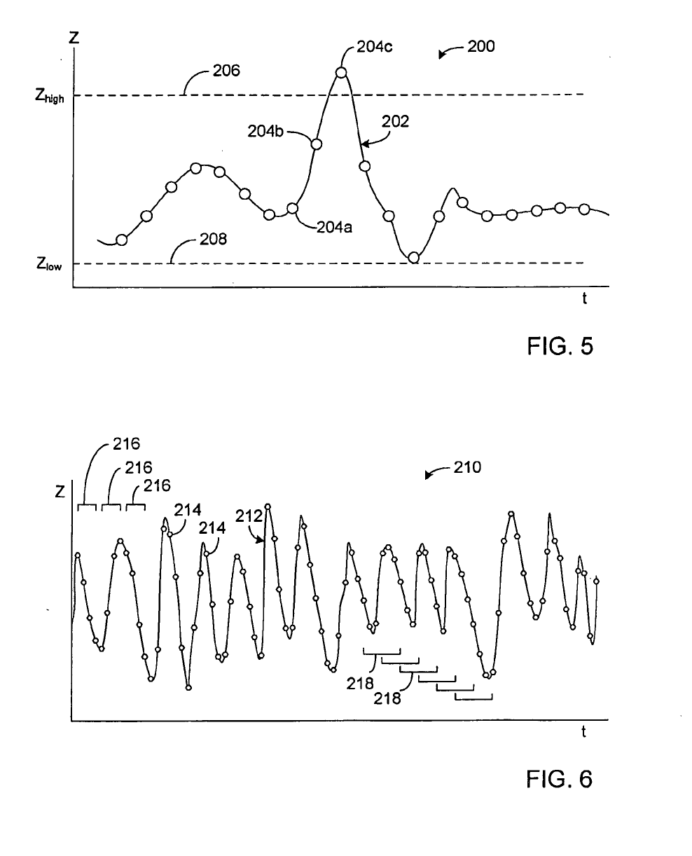

FIGS. 5-9 show some non-limiting examples of impedance data that can be monitored and processed to facilitate one or more operational features of an HVCS. FIG. 5 shows that in some embodiments, an impedance parameter such as impedance value Z associated with a high voltage lead can be sampled periodically. In an example impedance data 200, a plurality of sampled impedance values are depicted as data points 204. In some embodiments, a curve 202 can be obtained from the data points 204 (for example, by fitting) and represent time-dependence of impedance.

In some embodiments, one or more impedance threshold values can be set such that a condition can be triggered if a sampled impedance value goes beyond the set threshold value(s). For example, a Z_{high} threshold 206 can be set such that the example sampled value 204c exceeds the threshold 206. In such a situation, an action associated with the HVCS can be triggered. An example of how such threshold can be set, as well as example triggered actions, are described below in greater detail.

In another example, a Z_{low} 208 can be set such that if a sampled impedance value goes below the threshold (none shown in FIG. 5), an action associated with the HVCS can be triggered. An example of how such threshold can be set, as well as example triggered actions, are described below in greater detail.

FIG. 6 shows some non-limiting examples on variations in sampling techniques that can be utilized to sample impedance related parameters such as impedance value. In the example shown in FIG. 5, the impedance value can be sampled periodically, and each sampled value can be evaluated. In certain situations, the impedance values may fluctuate relatively rapidly, and it may be desirable to obtain average values that are less sensitive to such fluctuations. Thus, in an example impedance data 210 having impedance values 214 (that can be represented by a curve 212), average values can be formed among groups 216 of values 214. In some embodiments, the number of impedance values per group can be varied to achieve a desired averaging effect.

In some embodiments, as shown in FIG. 6, an averaging group of impedance values can overlap with its neighboring group. In certain sampling situations, such grouping and overlapping can allow smoothing of data. In some embodiments, such group size and/or the amount of overlap can be varied to achieve a desired smoothing effect. Other data averaging and/or smoothing techniques are also possible.

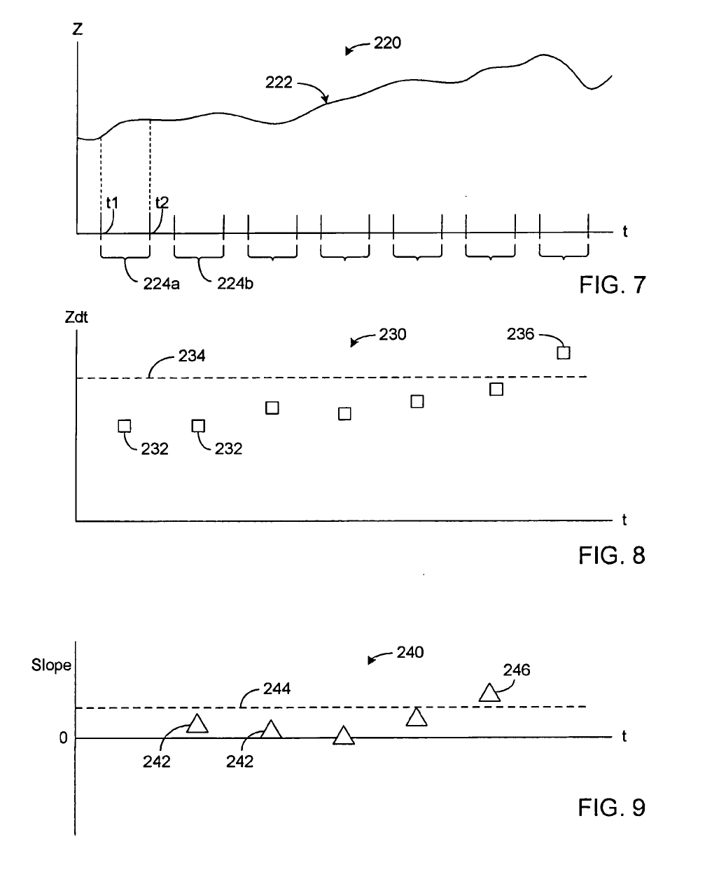

FIG. 7 shows an example impedance data 220 where impedance values (depicted as a curve 222) can be integrated over some time interval. For example, a plurality of integration time intervals are depicted as intervals 224. An example interval 224a is shown to begin at time t=t1 and end at t=t2, so that impedance is integrated to yield a Zdt value.

In some embodiments, such integration of impedance signal can be achieved by, for example, an integration circuit or via software using input sampled impedance values. The duration of the integration time interval(s) and/or any time intervals therebetween can be selected to achieve a desired range of Zdt values.

FIG. 8 shows an example Zdt data 230 that can result from the example Z data of FIG. 7. A plurality of Zdt data points 232 can represent integrated values of the corresponding intervals 224.

In some embodiments, as shown in FIG. 8, a threshold Zdt value 234 can be set to allow comparison with the Zdt data points. In the example shown in FIG. 8, a high threshold is shown; but it will be understood that a low threshold (not shown) can also be set. In the example, a Zdt data point 236 is depicted as exceeding the threshold value 234. Thus, a detection of such a Zdt value can trigger an action associated with the HVCS.

In some situations, use of Zdt may be less sensitive to Z signal fluctuations and provide a smoother trend indication of the impedance property of the high voltage lead. An example of how such threshold can be set, as well as example triggered actions, are described below in greater detail.

In some embodiments, various other quantities can be derived from Z and/or Zdt values. In a non-limiting example, FIG. 9 shows an example impedance data 240 where a trend of Zdt is plotted as a function of time. The data points 242 can represent a slope of the Zdt about the corresponding Zdt data points. For example, the first shown data point 242 can represent the slope of a line representing the first three data points 232 in FIG. 8.

In some embodiments, as shown in FIG. 9, a threshold slop 244 can be set to allow comparison with the slope data points. In the example shown in FIG. 9, a high threshold is shown; but it will be understood that a low threshold (not shown) can also be set. In the example, a slope data point 246 is depicted as exceeding the threshold value 244. Thus, a detection of such a slope value can trigger an action associated with the HVCS.

In some situations, use of such trend values may be less sensitive to Z signal fluctuations and provide a smoother trend indication of the impedance property of the high voltage lead. An example of how such threshold can be set, as well as example triggered actions, are described below in greater detail.

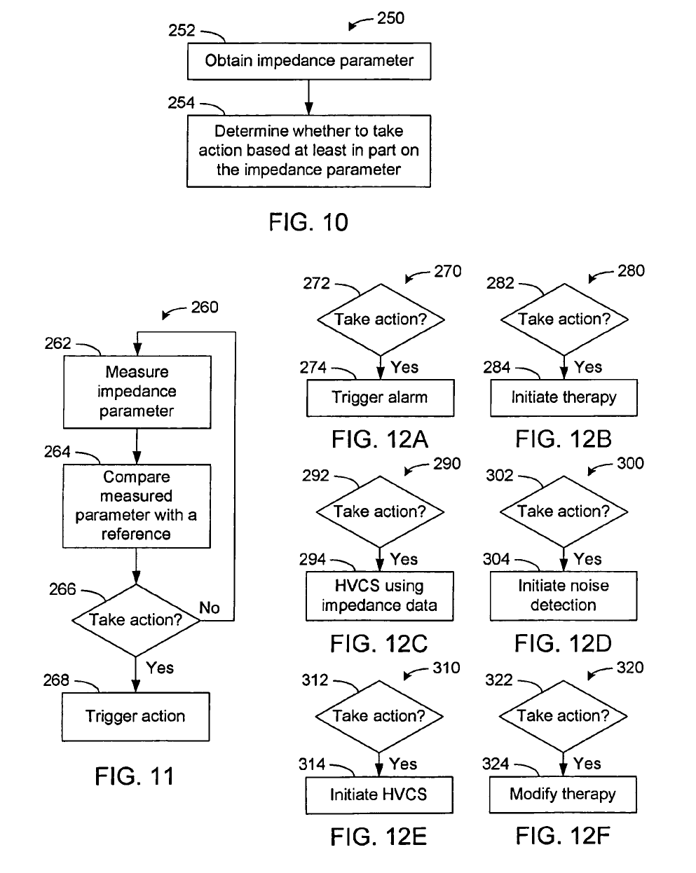

FIG. 10 shows that in some embodiments, a process 250 can be performed by the HVCS to utilize impedance data associated with the high voltage lead. In a process block 252, impedance parameter is obtained. In a process block 254, the process 250 determines whether to take action based at least in part on the impedance parameter.

FIG. 11 shows that in some embodiments, a process 260 can be an example of how the process 250 of FIG. 10 can be implemented. In a process block 262, an impedance parameter is measured. In a process block 264, the measured impedance parameter, or some quantity derived therefrom, is compared with a reference. Based on such comparison, the process 260 in a decision block 266 determines whether to take action associated with the operation of HVCS. If the answer is “No,” the process 260 can continue to monitor the impedance parameter (depicted by the loop-back to the process block 262). If the answer is “Yes,” the process 260 in a process block 268 triggers an action associated with the operation of HVCS.

FIGS. 12A-12F show non-limiting examples of HVCS associated actions that can be triggered based on the detection of an impedance parameter condition as described in reference to FIGS. 10 and 11. FIG. 12A shows that in some embodiments, a process 270 can be implemented, where the process 270 determines in a decision block 272 whether to trigger an action. If the answer is “Yes,” the process 270 is a process block 274 can trigger an alarm associated with the operation of the HVCS.

FIG. 12B shows that in some embodiments, a process 280 can be implemented, where the process 280 determines in a decision block 282 whether to trigger an action. If the answer is “Yes,” the process 280 is a process block 284 can initiate a therapy that includes the operation of the HVCS.

FIG. 12C shows that in some embodiments, a process 290 can be implemented, where the process 290 determines in a decision block 292 whether to trigger an action. If the answer is “Yes,” the process 290 is a process block 294 can perform one or more processes associated with the operation of the HVCS.

FIG. 12D shows that in some embodiments, a process 300 can be implemented, where the process 300 determines in a decision block 302 whether to trigger an action. If the answer is “Yes,” the process 300 is a process block 304 can initiate noise detection that can be a part of the HVCS.

FIG. 12E shows that in some embodiments, a process 310 can be implemented, where the process 310 determines in a decision block 312 whether to trigger an action. If the answer is “Yes,” the process 310 is a process block 314 can initiate the operation of HVCS itself.

FIG. 12F shows that in some embodiments, a process 320 can be implemented, where the process 320 determines in a decision block 322 whether to trigger an action. If the answer is “Yes,” the process 320 is a process block 324 can modify a therapy under the control of the HVCS.

Other configurations are possible.

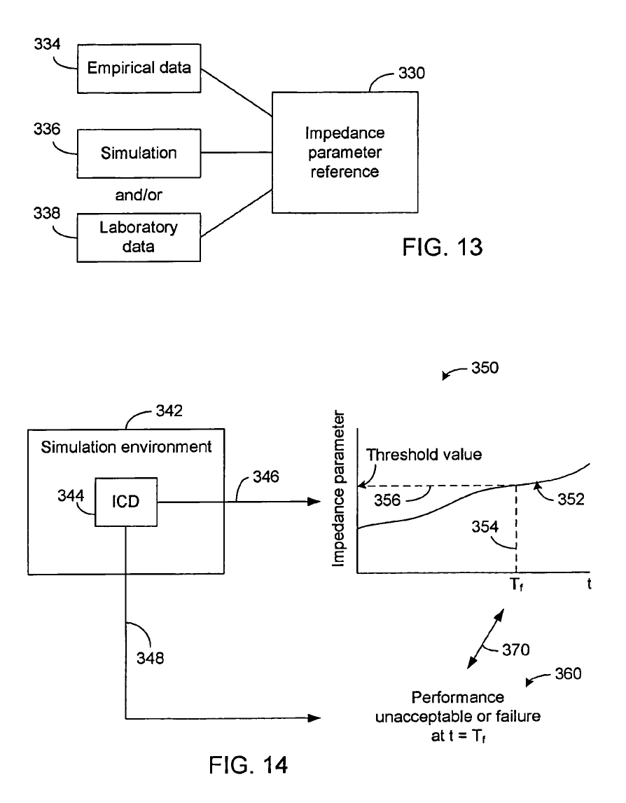

As shown in FIG. 11, a measured and/or derived value associated with impedance can be compared to a reference value. FIG. 13 shows an example of how such a reference can be formed. In some embodiments, an impedance parameter reference 330 can be formed based on input of data from one or more sources. Empirical data 334 can be used to provide one or more impedance parameters. For example, an implantable cardiac device removed from a patient can be assessed for failure modes, and corresponding impedance data for high voltage lead can be obtained.

In some embodiments, input for the reference 330 can be provided by a simulation component 336. Such simulation can be configured to predict various electrical properties (including impedance properties) associated with the high voltage lead. Such simulation data can be verified by data obtained empirically or by other studies.

In some embodiments, input for the reference 330 can be provides by laboratory data 338. For example, characteristic changes in one or more impedance parameters can be studied in simulated conditions in the laboratory. In the examples shows in FIGS. 7 and 8, the impedance Z and integrated impedance Zdt are depicted as increasing over time. Such increasing trend can be studies in the laboratory under controlled conditions, and one or more threshold conditions can be defines where the high voltage lead fails or becomes sufficiently undesirable.

In some embodiments, various combinations of the example inputs 334, 336, and 338 can be used to form the reference 330. Other inputs are also possible.

As described herein, impedance data measured or derived can be compared to a reference. In some embodiments, such a reference includes one or more threshold values. FIG. 14 shows an example of how such threshold value(s) can be obtained.

FIG. 14 shows an example study 350 that can be conducted, where an implantable cardiac device 344 is subjected to a simulated environment 342. While in such an environment, one or more impedance parameters can be measured and monitored (depicted as arrow 346). Also, one or more performance related parameters can be monitored (depicted as arrow 348). For example, the condition of the high voltage lead can be monitored for degradation.

Based on such performance monitoring, an unacceptable or failure condition 360 can be identified (for example, at time t=T_{f}). Such failure condition can be correlated (depicted as an arrow 370) with the monitored impedance data 352 by, for example, identifying the value of the impedance parameter (depicted as threshold value 356) corresponding to the failure time T_{f} (depicted as time 354).

A wide variety of variations, however, are possible. For example, additional structural and/or functional elements may be added, elements may be removed or elements may be arranged or configured differently. Similarly, processing steps may be added, removed, or ordered differently. Accordingly, although the above-disclosed embodiments have shown, described, and pointed out the novel features of the invention as applied to the above-disclosed embodiments, it should be understood that various omissions, substitutions, and changes in the form of the detail of the devices, systems, and/or methods shown may be made by those skilled in the art without departing from the scope of the invention. Consequently, the scope of the invention should not be limited to the foregoing description, but should be defined by the appended claims.

What is claimed is:

1. A system for differentiating noise from an arrhythmia of a heart, comprising:
a noise discriminator configured to receive an electrocardiogram (EGM) signal and to discriminate between an organized EGM signal and a chaotic EGM signal based at least in part on an impedance parameter associated with a lead that provides an electrical connection to the heart;
a signal analyzer configured to determine whether a chaotic signal is caused by a disturbance in the lead.

2. The system of claim 1, further comprising a high voltage delivery system configured to deliver a high voltage therapy signal to the heart if the EGM signal is an organized signal.

3. The system of claim 2, further comprising a high voltage confirmation system configured to adjust or terminate the high voltage therapy based on the impedance parameter.

4. The system of claim 3, wherein the signal analyzer is part of the high voltage confirmation system.

5. The system of claim 3, wherein the lead comprises a high voltage lead for delivering the high voltage signal to the heart.

6. The system of claim 3, wherein the impedance parameter comprises an impedance value associated with an electrical connection of the lead with the heart.

7. The system of claim 3, wherein the impedance parameter comprises an integrated value of impedance associated with an electrical connection of the lead with the heart.

8. The system of claim 1, wherein the signal analyzer determines whether the chaotic signal is caused by lead disturbance by comparing the impedance parameter with a known threshold value.

9. The system of claim 8, wherein the known threshold value comprises a threshold impedance value for the lead corresponding to a failure condition of the lead.

10. The system of claim 9, wherein the failure condition of the lead and the corresponding threshold impedance value are determined by providing a simulated operating condition of the lead in a laboratory.

11. An implantable cardiac device, comprising:
a high voltage device configured to deliver a therapy signal to a heart when triggered;
an electrical lead for connecting the high voltage device to the heart;
an impedance measurement component configured to measure an electrical impedance associated with the electrical lead; and
a processor configured to provide a command for operation of the high voltage device based at least in part on a parameter associated with the measured electrical impedance.

12. The system of claim 11, wherein the parameter comprises an electrical resistance.

13. The system of claim 11, wherein the parameter comprises an integrated value of electrical resistance over a period of time.

14. The system of claim 11, wherein the processor provides the command based on comparison of the parameter with a known reference value.

15. The system of claim 14, wherein the known reference value is stored in the implantable cardiac device, and obtained from a laboratory study that simulates degradation of the electrical lead.

16. The system of claim 15, wherein the known reference value is obtained by correlating an observed failure condition with a corresponding value of the parameter.

17. The system of claim 11, wherein the command comprises a termination command that terminates a process for delivering the therapy signal.

18. The system of claim 11, wherein the command comprises an adjustment command that adjusts the therapy signal.

19. A method for operating an implantable cardiac device, comprising:
measuring an impedance value associated with at least one of a plurality of electrical leads for a high voltage device configured to provide a therapy signal, wherein the plurality of electrical leads are configured to be connected to a heart and deliver the therapy signal to the heart; and
generating a command for operation of the high voltage device based at least in part on the measured impedance value.

20. A method for differentiating noise from an arrhythmia of a heart, comprising:
receiving an electrocardiogram (EGM) signal;
measuring an impedance parameter associated with a lead that provides an electrical connection to the heart; and
determining whether the EGM signal is an organized signal or a chaotic signal based at least in part on the measured impedance parameter.
